(19) United States
(12) Patent Application Publication
(10) Pub. No.: US 2013/0143831 A1
(43) Pub. Date: Jun. 6, 2013

(54) TOPICAL PHARMACEUTICAL COMPOSITION COMPRISING FLURBIPROFEN

(71) Applicants: Koral Embil, Istanbul (TR); Ray Figueroa, Medley, FL (US)

(72) Inventors: Koral Embil, Istanbul (TR); Ray Figueroa, Medley, FL (US)

(73) Assignee: EDKO PAZARLAMA TANITIM TICARET LIMITED SIRKETI, Istanbul (TR)

(21) Appl. No.: 13/807,324

(22) PCT Filed: Jun. 28, 2011
(86) PCT No.: PCT/GB11/51220

§ 371 Date: Dec. 28, 2012

(30) Foreign Application Priority Data
Jun. 29, 2010 (GB) 1010954

(51) Int. Cl. A61K 31/70; A61K 31/045; A61K 31/235; A61K 31/704; A61K 31/167; A61K 31/245; A61K 31/24; A61K 31/192

(52) U.S. Cl. CPC A61K 31/192; A61K 31/045; A61K 31/167; A61K 31/245; A61K 31/235; A61K 31/704
USPC 514/33; 514/570; 514/537

(57) ABSTRACT

The invention provides topical pharmaceutical compositions comprising flurbiprofen, or a pharmaceutically acceptable derivative thereof, in combination with a solubilising system which comprises at least one glycol ether and at least one glycol ester. These are suitable for treating any condition associated with pain, inflammation and/or stiffness, for example sub-dermal pain in the joints or soft tissue.

The present invention relates to topical flurbiprofen-containing compositions, to processes for their preparation and to their use as medicaments, for example in treating conditions associated with inflammation and/or pain. More particularly, the invention relates to such compositions in which the flurbiprofen active component remains solubilised, thereby providing good physicochemical stability and optimised transdermal delivery of the active.

Flurbiprofen (2-(3-fluoro-4-phenyl-phenyl)propanoic acid) is a well-known, non-steroidal anti-inflammatory drug (NSAID) which can be used to treat inflammation and/or pain. For example, it is widely used in relieving pain and treating inflammation associated with severe or chronic arthritis. In general, flurbiprofen is administered orally, e.g. in the form of tablets or capsules. However, oral administration of flurbiprofen-containing compositions can result in gastrointestinal irritation as well as systemic side effects such as liver and kidney problems. While topical flurbiprofen compositions are described in the patent literature, so far these have not been extensively used and are not widely available to patients. One known topical form of flurbiprofen is that marketed by Abbott under the trade name FROBEN™.

One particular problem associated with the development of topical flurbiprofen-containing compositions is that flurbiprofen has poor solubility in aqueous solvents, especially in water. A number of different carrier systems for topical administration of flurbiprofen have been proposed. For example, Japanese Patent application No. 56-154413 describes the preparation of a flurbiprofen composition in which a terpene or higher fatty acid ester is used to solubilise the active, then mixed with surfactants and water to form an oil-in-water emulsion.

U.S. Pat. No. 4,545,992 describes a composition in which flurbiprofen is incorporated into peppermint oil or certain esters of salicylic acid before emulsification into an aqueous base.

U.S. Pat. Nos. 4,393,076, 4,472,376 and 4,533,546 each disclose topical flurbiprofen compositions in which water and/or an alcohol are used to solubilise the active during the preparation of the topical composition. In each case, the pH must be controlled in order to control the stability and penetration characteristics of the topical compositions. The importance of pH control is also emphasised in U.S. Pat. No. 5,807,568 which discloses hydroalcoholic gel compositions with a pH in the range of about 2 to 5.5 and which maximise the flux of flurbiprofen through the skin. High levels of lower alcohols such as ethanol and propanol are used to solubilise the active prior to gelling.

The flurbiprofen compositions described above have at least one disadvantage. To the extent that these comprise oil-in-water emulsions these can experience stability problems, especially at elevated temperatures. In such emulsion systems where the flurbiprofen exists in insoluble suspensions (i.e. not fully dissolved in the carrier system), physical instability can lead to “breaking” of the formulation which produces a non-homogenous mixture of the different components and loss of therapeutic efficacy. This lack of complete solubility also results in re-crystallisation of the active which may lead to inaccurate dosing. Moreover, due to the aqueous nature of the known compositions, the pH must be carefully controlled in order to ensure that the active agent can penetrate the skin.

Other emulsion systems which have been proposed for the topical delivery of flurbiprofen include microemulsion suspensions of non-solubilised flurbiprofen. However, these can also experience physical instability due to non-homogeneous distribution of the active or lack of active uniformity.

In a similar manner, other vehicle systems that act as single phase suspension vehicles, such as aqueous hydroalcoholic gels for non-solubilised flurbiprofen particles, can experience non-homogeneous therapeutic dosage delivery as a result of physical instability of the system.

There thus exists a need for alternative topical flurbiprofen compositions, in particular those which are both chemically and physically stable whilst at the same time optimising the delivery of the active through the skin.

We have now developed certain novel formulations comprising flurbiprofen (or pharmaceutically acceptable derivatives thereof) which advantageously exhibit good skin penetrability and excellent storage stability. Such formulations have the additional advantage that these exhibit low (e.g. negligible) skin irritation.

More specifically, we have developed topical flurbiprofen compositions in which the active essentially remains in soluble form (i.e. is solubilised) by means of a unique solubilising system. Instead of relying on the presence of water and/or an alcohol as in many conventional formulations, the compositions of the present invention make use of the solvent and penetrating properties of the solubilising system to ensure that the active is fully solubilised (i.e. is maintained in solution) and capable of penetrating through the skin to the intended site of action (whether dermal or sub-dermal). The active is therefore delivered in the form of a solution of varied viscosities as opposed to an emulsion or suspension. To the extent that the active remains substantially in solution, it leaves no visible residue on the skin after application.

Typically, such compositions are substantially free from water (i.e. they are substantially anhydrous) and/or substantially free from any alcohol (e.g. lower alcohols such as ethanol and propanol). Particularly preferably, these are substantially free from both water and alcohol such that they have no readable pH measure.

By using a solvent system in which the active remains solubilised, the formulations exhibit excellent storage stability (e.g. 6 months or more, preferably in excess of 12, 18 or even 24 months at ambient temperature). The use of solvents which not only serve to adequately solubilise the active, but which also exhibit the necessary penetration enhancing properties, also ensures that the formulations demonstrate good skin penetrability.

Viewed from one aspect the invention thus provides a topical pharmaceutical composition comprising flurbiprofen, or a pharmaceutically acceptable derivative thereof, in combination with a solubilising system which comprises at least one glycol ether and at least one glycol ester.

Preferably, in such compositions the flurbiprofen is substantially solubilised. Solubilised flurbiprofen provides the advantage of immediate and uniform availability of the active drug molecules (since any active in crystalline form cannot be uniformly delivered through the skin unless it were to be uniformly delivered as a drug depot). The term “solubilised” is intended to mean that in the composition there is essentially an intimate dispersion or dissolution of the active agent such that few, if any, crystals of the active agent can be detected. As such, the active agent is considered to be substantially in “non-crystallised” form. Preferred compositions according to the invention are those which comprise less than 0.5 wt. % flurbiprofen (or flurbiprofen derivative) in crystalline form (based on the total amount of flurbiprofen in the composition), preferably less than 0.1 wt. %, e.g. less than 0.01 wt. %.

In general, the compositions according to the invention will not be in the form of oil-in-water or water-in-oil emulsions.

The flurbiprofen for use in the invention may comprise not only the conventionally used racemic mixture of the S and R enantiomers of 2-(3-fluoro-4-phenyl-phenyl)propanoic acid, but also the substantially pure enantiomers (e.g. comprising at least 90% by weight of the S or R enantiomer of flurbiprofen). Most typically, however, the racemic mixture will be used.

Suitable derivatives of flurbiprofen include the pharmaceutically acceptable salts and esters of flurbiprofen. Appropriate salts include base addition salts, for example sodium, potassium, calcium, magnesium, and zinc. Procedures for salt formation are conventional in the art.

The desired amount of flurbiprofen in the compositions will vary depending on the nature of the condition to be treated and can readily be determined by those skilled in the art. In general, the amount present may be up to 30% by weight, preferably from 0.5 to 20% by weight, more preferably 2 to 20% by weight, yet more preferably from 5 to 10% by weight, e.g. around 5% by weight.

Glycol ethers suitable for use in the compositions herein described include ethylene glycol monoethyl ether, diethylene glycol monoethyl ether, propylene glycol monoethyl ether and dipropylene glycol monoethyl ether. Although a mixture of glycol ethers may be used, a single glycol ether is preferred. Particularly preferred is diethylene glycol monoethyl ether or DGME (also known as ethoxydiglycol). DGME is a pharmaceutical grade transparent liquid (MW 134.2) with unique solubilising properties. It has the ability not only to solubilise both hydrophilic and hydrophobic materials, but also has penetration enhancing properties. It is marketed as a highly purified liquid under the trade name Transcutol (Gattefosse s.a., Saint Pres Cedex, France).

Glycol esters for use in the invention are typically di- or mono-esters of propylene glycol. However, other glycol esters such as esters of ethylene glycol may also be employed. Preferred propylene glycol esters are the esters of propylene glycol and saturated or unsaturated fatty (e.g. C₁₀₋₃₀) acids such as butyric, caprylic, capric, lauric, stearic, arachidic, behenic acids, etc. Particularly preferred are propylene glycol dipelargonate (DPPG™, Gattefosse), propylene glycol dicaprylocaprate (Labrafac™ PG, Gattefosse), propylene glycol monolaurate (Lauroglycol™ 90, Gattefosse), propylene glycol laurate (Lauroglycol™ FCC, Gattefosse), propylene glycol monocaprylate (Capryol 90™, Gattefosse), propylene glycol caprylate (Capryol™ PGMC, Gattefosse). Propylene glycol diesters, such as propylene glycol dipelargonate, are especially preferred and have the advantage that these also provide emollient properties.

In a preferred embodiment, the solubilising system may further comprise one or more additional co-solvents, preferably co-solvents having skin penetration-enhancing properties. Suitable co-solvents include glycols such as propylene glycol, 2-pentylene glycol, ethoxydiglycol; N-methylpyrrolidone, liquid polyethylene glycols such as PEG-200 (PEG-4), PEG-300 (PEG-6), PEG-400 (PEG-8) and PEG-600 (PEG-12); methoxypolyethylene glycol 550, polyglycol 300 (PEG-6); apricot kernel oil, propylene glycol monocaprylate (Capryol 90™), propylene glycol caprylate (Capryol™ PGMC), polyglyceryl diisostearate (Plurol® Diisostearate), polyglyceryl oleate (Plurol® Oleique CC497), polyglyceryl 6-distearate (Plurol® Stearique WL 1009), isostearyl isostearate, octyldodecyl myristate (MOD™), medium chain triglycerides such as Labrafac™ lipophile WL 1349, propylene glycol dipelargonate (DPPG™), propylene glycol dicaprylocaprate (Labrafac™ PG), propylene glycol monolaurate (Lauroglycol™ 90), propylene glycol laurate (Lauroglycol™ FCC); polyoxylglycerides such as oleoyl macrogolglycerides (Labrafil® M1944CS), linoleoyl macrogolglycerides (Labrafil® M2125CS) and caprylocaproyl macrogolglycerides (Labrasol®); diethylene glycol monoethyl ether (Transcutol® P).

The polyoxylglycerides (also known as macrogolglycerides or PEG glycerides) are particularly preferred for use as a co-solvent in the solubilising systems herein described and have the advantage that these also function as skin penetration enhancing agents. Polyoxylglycerides are mixtures of monoesters, diesters, and triesters of glycerol and monoesters and diesters of polyethylene. They are produced by partial alcoholysis of unsaturated oils, mainly containing triglycerides of fatty acids, with polyethylene glycol, by esterification of glycerol and polyethylene glycol with fatty acids, or as a mixture with glycerol esters and ethylene oxide condensate with fatty acids of the unsaturated oils. Particularly preferred are caprylocaproyl polyoxylglycerides such as PEG-8-caprylic capric glycerides (for example Labrasol®) which is the polyethylene glycol derivative of the mono- and diglycerides derived from caprylic and capric acids with an average of 8 moles of ethylene oxide. Other suitable polyoxylglycerides include PEG-6-caprylic/capric glyceride, Softigen 767 and the Acconon series of polyethoxylated glycerides marketed by Abitec (e.g. Acconon C-30, C-80, C-400, etc.)

A further example of a co-solvent which may also be present in the formulations herein described is dimethyl isosorbide. This may be used in an amount of from 0.1 to 20 wt. %, preferably 0.5 to 15 wt. %, e.g. 1 to 10 wt. %. Dimethyl isosorbide not only has excellent solvent properties but is also capable of enhancing the delivery of the active ingredient(s) through the skin. Particularly preferred for use in the invention is Super Refined Arlasolve DMI (Dimethyl Isosorbide) which is commercially available from Croda.

Particularly preferred for use as an additional co-solvent in the solubilising systems herein described is a polyoxylglyceride, optionally in combination with dimethyl isosorbide.

Other conventional skin penetration enhancers may also be provided in the compositions in accordance with the invention. Examples of suitable skin penetration enhancing agents include propylene glycol laurate, propylene glycol monolaurate, glyceryl esters (e.g. glyceryl monooleate), propylene glycol monocaprylate, isopropyl myristate, sodium lauryl sulphate, dodecyl pyridinium chloride, oleic acid, propylene glycol, nicotinic acid esters, hydrogenated soya phospholipids, essential oils, terpenes, alpha-tocopherol, polyethylene glycol succinate, Tween 80 and other surfactants, and dimethylsulphoxide (DMSO).

Preferably the compositions of the invention are provided in the form of gels, preferably semi-solid gels, and therefore will generally comprise at least one gelling agent. Where the product is provided in the form of a gel, this will typically be substantially free from water.

Any pharmaceutically acceptable gelling agent may be used in the formulations herein described, for example cellulose derivatives such as hydroxyethyl cellulose, hydroxypropylcellulose, hydroxypropyl methyl cellulose, carboxymethylcellulose, sodium carboxymethylcellulose; natural gums; chitin; chitosan; alginates; collagens; gelatin; pectin; polymerised acrylic acids either neutralised or un-neutralised such as carbomers and polycarbophils, etc. A particularly preferred gelling agent is hydroxypropylcellulose (HPC) which is available from Hercules, Inc. as KLUCEL HF. Other known gelling agents may also be used in the invention provided that they are compatible with the solubilising system.

The amount of gelling agent to be included in the compositions can readily be determined by those skilled in the art. In general, this will be present in an amount of up to 5 wt. %, preferably up to 3 wt. %, more preferably up to 2 wt. %, e.g. about 1.25 wt. %. In general the viscosities of the formulations may be up to 100,000 cps, preferably in the range 10,000 to 50,000 cps, yet more preferably 20,000 to 40,000 cps, e.g. about 25,000 cps at 20° C.

One particularly preferred aspect of the invention is that the compositions herein described should be substantially anhydrous, i.e. substantially free from water. By “substantially free” from water, it is intended that the compositions should comprise less than 10 wt. %, preferably less than 5 wt. %, more preferably less than 3 wt. %, e.g. less than 1 wt. % water.

The compositions herein described are also preferably substantially free from volatile organic solvents, such as alcohols (e.g. lower alcohols such as ethanol and propanol). By “substantially free” from any volatile organic solvent (e.g. an alcohol), it is intended that the compositions should comprise less than 10 wt. %, preferably less than 5 wt. %, more preferably less than 3 wt. %, e.g. less than 1 wt. % volatile organic solvent (e.g. alcohol).

Particularly preferred are those compositions which are substantially free from (e.g. free from) both water and volatile organic solvents such as alcohols.

The particular combination of components used in the compositions of the invention optimises skin penetration whilst minimising skin irritation. Nevertheless, further excipients, such as emollients and moisturisers may be present. Examples of other ingredients which may be present in the compositions of the invention include, but are not limited to, preservatives (e.g. antimicrobials or antifungals such as methyl paraben or propyl paraben); anti-oxidants; stabilizers; chelating agents such as EDTA; etc.

For example, anti-oxidants such as vitamin E, ascorbyl palmitate or butylated hydroxytoluene, may be added to the compositions to prevent degradation of the components. Anti-oxidants may be present in amounts from 0.01 to 5.0 wt. %, preferably 0.01 to 0.05 wt. %.

Other components which may be present include those having a local anaesthetic effect on the skin. One example of such a component is menthol which provides a cooling effect on the surface of the skin. Menthol may also serve to increase skin penetration of the formulation.

Other local anaesthetics which may be present in the formulations include the amide-type anaesthetics such as aptocaine, bupivacine, butanilicaine, carticaine, cinchocaine, clibucaine, ethyl parapiperidinoacetyl-aminobenzoate, etidocaine, lidocaine, mepivacaine, oxethazaine, prilocaine, pyrrocaine, ropivacaine, tolycaine or vadocaine, or any mixture thereof. Local anaesthetics of the p-aminobenzoic acid ester type such as benzocaine may also be used. Lidocaine, benzocaine and prilocaine are particularly preferred. Any of these substances may be used in the form of a salt.

Where present, any local anaesthetic (e.g. menthol, lidocaine, benzocaine or prilocaine) may be provided in an amount of up to 10 wt. %, preferably 0.05 to 5 wt. %, e.g. 0.1 to 3 wt. %.

The desired amount of the solubilising systems herein described (and also the amount of each component present within such systems) will depend on a number of factors, including the desired concentration of the drug and may be varied as needed. Typically, the solubilising system will comprise up to 98 wt. % of the total formulation. For example, this may comprise from 60 to 95 wt. %, more preferably from 80 to 95 wt. %, e.g. about 90 wt. % of the total formulation.

The major component of the solubilising system will generally be the glycol ether. This may be present at concentrations of up to 95% by weight, preferably 20 to 80% by weight, more preferably 50 to 70% by weight, e.g. about 65% by weight (based on the total weight of the formulation).

The glycol ester may be present at concentrations of up to 50% by weight of the total formulation, preferably 5 to 40% by weight, more preferably 10 to 30% by weight, e.g. about 25% by weight.

When present, any additional co-solvent (or co-solvents) will be present in a concentration of 1 to 20% by weight, preferably 1 to 15% by weight (based on the total weight of the formulation). Where the co-solvent is a polyoxylglyceride (for example the penetration enhancer Labrasol®), this will be present in a concentration of 1 to 10% by weight, preferably around 5% by weight (based on the total weight of the formulation).

An example of a particularly preferred composition in accordance with the invention is one comprising about 5 wt. % flurbiprofen; about 65 wt. % glycol ether (e.g. diethylene glycol monoethyl ether); about 25 wt. % glycol ester (e.g. propylene glycol dipelargonate); about 5 wt. % additional co-solvent (e.g. PEG-8-caprylic capric glycerides); and about 1 wt. % gelling agent (e.g. HPC). In this formulation, 10 wt. % of the glycol ether (based on the total weight of the formulation) may be replaced by dimethyl isosorbide.

Other known anti-inflammatory agents (e.g. topical analgesics) may also be present in the compositions herein described. Where present, these may be provided in amounts of up to 20 wt. %, e.g. from 0.01 to 20 wt. %. One particularly preferred agent is capsaicin which acts as a natural anti-inflammatory agent. Capsaicin is extractable from peppers and contains the active component 8-methyl-N-vanillyl-6-nonenamide. A further example of an anti-inflammatory agent which may be present is thiocolchicoside and its pharmaceutically acceptable salts; thiocholchicoside is a natural glycoside muscle relaxant having anti-inflammatory and analgesic effects. It is preferred that thiocholchicoside will be present in an amount of up to 5 wt. %. e.g. 0.1 to 5 wt. %.

The compositions herein described can be prepared by methods conventionally known and used in the art for the manufacture of creams, gels, etc. One suitable method for the preparation of a composition in the form of a semi-solid gel includes the step of mixing the glycol ether with the active flurbiprofen (and optionally a further anti-inflammatory agent) to form a clear solution to which the required amount of gelling agent may be added. If necessary, complete hydration of the gelling agent may be accelerated either by increased speed of stirring and/or by heating the mixture. However, if heat is applied this should be moderate heat only, e.g. not exceeding 40° C., preferably in the range 30-35° C. Once a lump-free gel is formed, this is added with mixing to the remaining components of the solubilising system (i.e. the glycol ester and any additional co-solvent or co-solvents) to form a homogenous gel following cooling to ambient temperature.

Viewed from a further aspect the invention thus provides a process for the preparation of a flurbiprofen composition as herein described, said process comprising the step of dissolving flurbiprofen, or a pharmaceutically acceptable salt thereof, in a solubilising system as herein described.

Preferably, the particular combination of components which forms the basis of the invention provides a composition in the form of a clear, semi-solid gel wherein the drug is in solution. Advantageously, such compositions leave no visible residue on the skin after application. The compositions achieve “stable” solubility without recrystallisation of the drug. Moreover, the particular combination of excipients which form the basis of the present invention confers the advantage that the flurbiprofen may be solubilised in the absence of water and/or alcohol. The compositions therefore allow a high degree of penetration via topical administration and also, because of their hydrophobic nature, they provide drug-delivery in a more steady state of diffusion since there is little to no evaporation of volatiles to disrupt the permeation characteristics.

The topical flurbiprofen compositions of the present invention can be used for treating a variety of indications characterised by one or more of the following symptoms: pain, inflammation and stiffness. In particular, these may be used in treating sub-dermal pain in the joints or soft tissue, e.g. muscular or tendon pain, pain in scar tissue or at surgical incision sites, joint pains, chest pains, back pains, bursal pains (e.g. associated with bursitis). Examples of such indications include osteoarthritis of superficial joints, such as the knee, ankle, wrist and elbow; rheumatism; acute musculoskeletal injuries and/or bruising; muscular cramp; strains; sprains; periarthritis; epicondylitis; tendinitis; bursitis; tenosynovitis; tennis elbow; back strain; lumbago; sciatica; neuralgia and fibrositis. It is envisaged that the compositions herein described will be of particular use in treating (e.g. reducing or eliminating) muscular pain, especially pain associated with arthritic conditions such as rheumatoid arthritis.

Viewed from a further aspect the invention thus provides a composition in accordance with the invention for use in medicine, in particular for treating a condition associated with at least one of the following symptoms: pain, inflammation and stiffness.

Viewed from a further aspect the invention provides the use of a composition as herein described in the manufacture of a medicament for use in treating a condition associated with at least one of the following symptoms: pain, inflammation and stiffness, for example in the treatment of pain. Preferably the medicament is for topical application.

In a still further aspect the invention provides a method of treatment of the human or non-human (in particular mammalian) animal body to combat a condition associated with at least one of the following symptoms: pain, inflammation and stiffness, said method comprising topically applying to the skin of said body a composition as herein described.

The compositions herein described may also be used as a chemopreventive agent, for example in the prevention or treatment of UV light-induced skin cancers or pre-cancerous lesions. As used herein, the term “chemopreventive agent” is intended to encompass any agent which reverses, suppresses or prevents cancer. The compositions are particularly suitable for the prevention or treatment of non-melanoma skin cancers such as squamous and basal cell carcinomas. When used for the purpose of preventing the occurrence of non-melanoma skin cancers, the compositions according to the invention may be applied regularly to the skin of the patient, in particular to the areas of the face, neck and arms which tend to receive the highest level of exposure to the sun.

Due to the known anti-proliferative effects of flurbiprofen, the compositions according to the invention also find use in the prevention or treatment of a range of disorders in which the skin exhibits abnormal proliferation. Such conditions include psoriasis, actinic keratoses, hyperkeratosis, seborrheic dermatitis, etc.

A further use for the compositions herein described is as an anti-microbial, for example as an anti-fungal, anti-bacterial or anti-protozoal agent. For example, these may be topically applied for use in treating superficial fungal, yeast or bacterial infections. When used in this way, the additional anti-inflammatory activity of the flurbiprofen helps in relieving any skin inflammation which may be associated with the infection.

In a further aspect the invention thus provides a composition in accordance with the invention for use as a chemopreventive agent; for use in the prevention or treatment of a hyperproliferative disorder; or for use as an anti-microbial. Corresponding methods of medical treatment also form further aspects of the invention.

Preferably, the compositions of the invention are semi-solid gels which, in use, are topically applied to the surface of the skin. Following application to the skin, these may be occluded by means of a film or barrier which may be permeable, semi-permeable or impermeable. Occlusion may in some cases serve to enhance the rate and/or degree of penetration of the active across the skin. Alternatively the gels may remain non-occluded on the surface of the skin. Gels incorporating the active agent may also be formulated into transdermal delivery systems or devices, such as patches for application to the skin. Although the compositions will preferably take the form of a gel (i.e. these contain at least one gelling agent), these may also be formulated as a lotion, cream or ointment.

The compositions herein described are applied topically to the skin which should be clean and preferably cleansed before application. Administration may be intermittent in time, e.g. four times daily, twice daily, daily, etc., depending on the nature of the condition to be treated.

The invention is further illustrated by way of the following non-limiting Examples and the accompanying FIGURE in which:

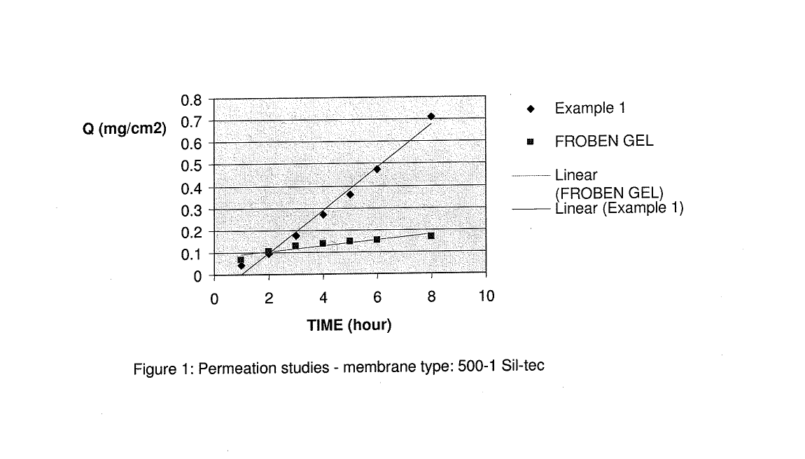

FIG. 1—shows the permeation across a Sil-tec membrane of the formulation according to Example 1 compared to Froben gel. For Example 1: y=0.0962x−0.0952 and R²=0.9861; for Froben gel: y=0.0126x+0.00765 and R²=0.8634.

### EXAMPLE 1

### Topical Gel

| INGREDIENT | % w/w |
|---|---|
| Ethoxydiglycol, NF grade (Diethylene glycol monoethyl ether, | 64.70 |
| Transcutol ® P, Gattefosse) | |
| Butylated Hydroxytoluene (BHT, Eastman) | 0.05 |
| Flurbiprofen USP (Selectchemie) | 5.00 |
| Hydroxypropylcellulose, NF (Klucel HF Pharm., Hercules) | 1.25 |
| Propylene Glycol Dipelargonate (DPPG, Gattefosse) | 24.00 |
| PEG-8-Caprylic Capric Glycerides (Labrasol ®, Gattefosse) | 5.00 |

### Preparation:

1. With mixing, BHT was slowly added to Transcutol. Further mixing was then carried out to dissolve solids.
2. Once a complete solution of BHT was obtained, Flurbiprofen was slowly sprinkled in and mixed to solubilise solids (a transparent clear solution was formed at this point).
3. While mixing at room temperature (adjusting the speed as necessary), the required amount of hydroxypropyl cellulose (HPC) was sprinkled into the solution. The HPC was hydrated to form a lump-free gel (optionally, complete polymer hydration can be accelerated either by increasing mechanical mixing speed and/or warming the mixture in the range of 30-35° C. while a vigorous to moderate mechanical mixing is applied).
4. As soon as a lump-free gel was formed (no “fish eyes” are seen at this point), the temperature of the mixture was maintained at 30-35° C. (main batch).
5. Meanwhile, a mixture of DPPG and Labrasol was warmed to 30-35° C.
6. The DPPG-Labrasol mixture was added to the main batch with moderate to vigorous mixing.
7. Whilst cooling the resulting mixture to room temperature, mixing was performed to form a homogeneous, lump-free, gel.

### EXAMPLE 2

### Topical Gel

| INGREDIENT | % w/w |
|---|---|
| Diethylene glycol monoethyl ether, Transcutol ® P, Gattefosse) | 54.70 |
| Butylated Hydroxytoluene (BHT, Eastman) | 0.05 |
| Flurbiprofen USP (Selectchemie) | 5.00 |
| Hydroxypropylcellulose, NF (Klucel HF Pharm., Hercules) | 1.25 |
| Propylene Glycol Dipelargonate (DPPG, Gattefosse) | 24.00 |
| Dimethyl Isosorbide (Super Refined Arlasolve DMI, Croda) | 10.00 |
| PEG-8-Caprylic Capric Glycerides (Labrasol ®, Gattefosse) | 5.00 |

### Preparation:

1. With mixing, BHT was slowly added to Transcutol. Further mixing was then carried out to dissolve solids.
2. Once a complete solution of BHT was obtained, Flurbiprofen was slowly sprinkled in and mixed to solubilise solids (a transparent clear solution was formed at this point).
3. While mixing at room temperature (adjusting the speed as necessary), the required amount of hydroxypropyl cellulose (HPC) was sprinkled into the solution. The HPC was hydrated to form a lump-free gel (optionally, complete polymer hydration can be accelerated either by increasing mechanical mixing speed and/or warming the mixture in the range of 30-35° C. while a vigorous to moderate mechanical mixing is applied).
4. As soon as a lump-free gel was formed (no “fish eyes” are seen at this point), the temperature of the mixture was maintained at 30-35° C. (main batch).
5. Meanwhile, a mixture of DPPG, Arlasolve and Labrasol was warmed to 30-35° C.
6. The DPPG-Arlasolve-Labrasol mixture was added to the main batch with moderate to vigorous mixing.
7. Whilst cooling the resulting mixture to room temperature, mixing was performed to form a homogeneous, lump-free, gel.

### EXAMPLE 3

### Diffusion Study

Preparation of buffer solution (pH 7.4) (USP): 50 ml 0.2 M potassium dihydrogen phosphate (KH₂PO₄) solution and 39.1 ml 0.2 M sodium hydroxide (NaOH) solution are added to a 200 ml volumetric flask. This is diluted to the desired volume with deionized water. The pH of the solution is 7.4.

The permeation of the formulation of Example 1 compared to Froben gel across a Sil-tec membrane is measured under the following test conditions:

Speed of stirrbar: 400 rpm

Sampling points: 1, 2, 3, 4, 5, 6 and 8 hours

Temperature is set to 3° C.±1° C.

The results are shown in accompanying FIG. 1. From this FIGURE it can be seen that:
1. The permeation of flurbiprofen from the formulation of Example 1 across a Sil-tec membrane increases at a rate of 0.0962 mg/cm² over a period of 10 hours.
2. The permeation of flurbiprofen from Froben Gel across a Sil-tec membrane increases at a rate of 0.0126 mg/cm² over a period of 10 hours.
3. The rate of permeation of flurbiprofen from the formulation of Example 1 is 7.6 times faster than from Froben Gel.
4. The total amount of flurbiprofen permeated from the formulation of Example 1 across a 1.77 cm² Sil-tec membrane is 2.12 mg/cm² over a period of 10 hours.
5. The total amount of flurbiprofen permeated from Froben Gel across a 1.77 cm² Sil-tec membrane is 0.90 mg/cm² over a period of 10 hours.
6. The total amount of flurbiprofen permeated from the formulation of Example 1 is 2.3 times more than from Froben Gel.

1. A topical pharmaceutical composition comprising flurbiprofen, or a pharmaceutically acceptable derivative thereof, in combination with a solubilising system which comprises at least one glycol ether and at least one glycol ester.

2. A composition as claimed in claim 1 which comprises less than 0.5 wt. % flurbiprofen in crystalline form, preferably less than 0.1 wt. %, e.g. less than 0.01 wt. % (based on the total amount of flurbiprofen in the composition).

3. A composition as claimed in claim 1 which is substantially free from water.

4. A composition as claimed in claim 1 which is substantially free from volatile organic solvents.

5. A composition as claimed in claim 1 wherein the flurbiprofen, or derivative thereof, is present in an amount of up to 30% by weight, preferably from 0.5 to 20% by weight, more preferably 2 to 20% by weight, yet more preferably from 5 to 10% by weight, e.g. around 5% by weight.

6. A composition as claimed in claim 1 wherein said glycol ether is selected from ethylene glycol monoethyl ether, diethylene glycol monoethyl ether, propylene glycol monoethyl ether and dipropylene glycol monoethyl ether.

7. A composition as claimed in claim 6 wherein said glycol ether is diethylene glycol monoethyl ether.

8. A composition as claimed in claim 1 wherein said glycol ester is a di- or mono-ester of propylene glycol, preferably an ester of propylene glycol and saturated or unsaturated fatty acids.

9. A composition as claimed in claim 8 wherein said glycol ester is propylene glycol dipelargonate.

10. A composition as claimed in claim 1 wherein said solubilising system further comprises one or more additional co-solvents.

11. A composition as claimed in claim 10 wherein said co-solvent is a polyoxylglyceride.

12. A composition as claimed in claim 11 wherein said co-solvent is a caprylocaproyl polyoxylglyceride, preferably PEG-8-caprylic capric glycerides.

13. A composition as claimed in claim 1 which further comprises at least one gelling agent.

14. A composition as claimed in claim 1 which further comprises at least one local anaesthetic, preferably menthol, lidocaine, benzocaine or prilocaine.

15. A composition as claimed in claim 1 which further comprises one or more additional anti-inflammatory agents.

16. A composition as claimed in claim 15 wherein said anti-inflammatory agent is capsaicin or thiocolchicoside.

17. (canceled)

18. (canceled)

19. A method of treatment of the human or non-human animal body to combat a condition associated with at least one of the following symptoms: pain, inflammation and stiffness, said method comprising topically applying to the skin of said body a composition as claimed in claim 1.

20. (canceled)

21. (canceled)

22. A method of treatment of the human or non-human animal body to combat a UV light-induced skin cancer or pre-cancerous lesion; a hyperproliferative skin disorder; or a superficial skin infection, said method comprising topically applying to the skin of said body a composition as claimed in claim 1.
